(19) United States
(12) Patent Application Publication
(10) Pub. No.: US 2008/0129993 A1
(43) Pub. Date: Jun. 5, 2008

(54) WINDOWLESS FIBER OPTIC RAMAN SPECTROSCOPY PROBES

(76) Inventors: James F. Brennan, Matthews, NC (US); Jon Nazemi, Cambridge, MA (US); Eric Marple, Laxahatchee, FL (US)

Correspondence Address: DIAMOND LAW OFFICE LLC, 1605 JOHN STREET, SUITE 102, FORT LEE, NJ, 07024, US

(21) Appl. No.: 11/876,899

(22) Filed: Oct. 23, 2007

Related U.S. Application Data
(60) Provisional application No. 60/853,427, filed on Oct. 23, 2006.

(51) Int. Cl. G01J 3/44; A61B 6/00

(52) U.S. Cl. 356/301; 600/478

(57) ABSTRACT

The invention provides windowless fiber optic Raman probes that are directly or indirectly enclosed by one or more materials having a very low Raman signal in a desired wavenumber range that is used to evaluate a sample. In one embodiment, the very low Raman signal material is a polymer, such as a fluoropolymer, that has a very low Raman cross-section in the high wavenumber Raman region (approximately 2,600 to 3,200 cm⁻¹) which has been shown to be useful in evaluating blood vessels for atherosclerotic lesions. Still another embodiment of the invention provides windowless side-viewing intravascular catheters for evaluating lumen walls such as blood vessel walls.

This application claims the benefit of U.S. provisional application Ser. No. 60/853,427 filed Oct. 23, 2006.

### FIELD OF THE INVENTION

The invention relates to fiber optic catheters, and probes generally, housed in materials with very low Raman scattering cross sections.

### BACKGROUND OF INVENTION

Optical fibers permit the collection of Raman scattered light from otherwise inaccessible locations and routing of the collected signal to spectrometer systems for analysis. In biomedical applications, these optical fibers are generally enclosed in polymer tubing to protect the optics in the optical probe and to avoid contaminating the biomaterial. In previous realizations of Raman optical fiber probes, the excitation light was directed to a sample and the resulting scattered light was collected from a sample by placing the optical fibers and/or its collection optics in proximity with the material to be investigated. One generally avoided having foreign materials between the optical fiber collection optics and the sample.

In cardiovascular applications, it is desirable to enclose the fiber optic assembly in polymer tubing to protect the probe optics and to provide a smooth catheter surface to navigate delicate arteries. In prior Raman probes, an optical window interrupting the tubing has generally been provided to permit transmission and collection of light via the probe.

U.S. Pat. No. 6,522,913 discloses systems and methods for visualizing tissue during diagnostic or therapeutic procedures that utilize a support structure that brings sensors into contact with the lumen wall of a blood vessel, and is incorporated by reference herein in its entirety

U.S. Pat. No. 6,701,181 discloses multi-path optical catheters, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,949,072 discloses devices for vulnerable plaque detection, and is incorporated by reference herein in its entirety.

U.S. Publication No. 2002/0183622 discloses a fiber-optic apparatus and method for the optical imaging of tissue samples, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

U.S. Publication No. 2004/0204651 discloses infrared endoscopic balloon probes, and is incorporated by reference herein in its entirety.

U.S. Publication No. 2004/0260182 discloses intraluminal spectroscope devices with wall-contacting probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0075574 discloses devices for vulnerable plaque detection that utilize optical fiber temperature sensors, and is incorporated by reference herein in its entirety.

U.S. Publication No. 2005/0165315 discloses a side-firing fiber-optic array probe, and is incorporated by reference herein in its entirety.

U.S. Publication No. 2006/0139633 discloses the use of high wavenumber Raman spectroscopy for evaluating tissue, and is incorporated by reference herein in its entirety.

### SUMMARY OF INVENTION

The present invention provides windowless fiber optic Raman spectroscopy probes such as windowless intravascular catheters for evaluating blood vessel walls. The fiber optic probes of the invention include one or more optical fibers housed in material(s) having a very low Raman scattering cross-section in the wavenumber region used for analysis of a target and being adequately transparent to excitation light delivered via the fiber optics to the target and to Raman-scattered light (inelastically scattered light) collected from the irradiated target in the desired wavenumber range.

One embodiment of the invention provides a fiber optic Raman spectroscopy probe that includes: an optical fiber assembly having a viewing portion for transmitting and receiving light, wherein at least the viewing portion of the optical fiber assembly is enclosed in a material, such as a polymeric material, having an at least substantially non-discemable Raman scattering signal in one or more preselected wavenumber regions used for analysis of a target and being adequately transparent to excitation light delivered via the optical fiber assembly to the target and to Raman-scattered light collected from the irradiated target in the preselected wavenumber range by the optical fiber assembly.

A related embodiment of the invention provides an intravascular diagnostic catheter that includes at least one Raman spectroscopy probe as described.

A more particular intravascular catheter embodiment of the invention for optically interrogating a blood vessel wall by Raman Spectroscopy includes: multiple rod elements along a central shaft of the catheter and extendable radially outward toward a blood vessel wall from an unextended configuration closer to the longitudinal axis of the catheter, wherein the rod elements each comprise an optical fiber assembly having a viewing portion for transmitting and receiving light from the vessel wall lateral to the axis of the catheter while the rod-elements contact or are near the wall, wherein each of the optical assemblies is in optical communication with a light source for illuminating the vessel wall and/or a detector for detecting light received from the vessel wall. Again, at least the viewing portion of the optical fiber assemblies are enclosed in a material, such as a polymeric material, having an at least substantially non-discernable Raman scattering signal in one or more preselected wavenumber regions used for analysis of a target and being adequately transparent to excitation light delivered via the optical fiber assembly to the target and to Raman-scattered light collected from the irradiated target in the preselected wavenumber range by the optical fiber assembly.

The optical fiber assemblies of the probes and catheter probes may include one or more optical fibers. The preselected wavenumber region may, for example, be in the range of approximately 2,600 to 3,200 cm⁻. The enclosure material may, for example, include or consist of polymer material that at least substantially does not include carbon-hydrogen bonds, such as a fluoropolymer, for example, polytetrafluoroethylene (PTFE), fluorinated ethylene-propylene (FEP) and perfluoroalkoxy polymer resin (PFA). The enclosure material may, for example, include or consist of an amorphous fluoropolymer, such as Teflon AF™ (DuPont, Wilmington, Del.).

Another embodiment of the invention provides a method for evaluating a blood vessel, such as an artery, in order to determine the presence, absence and/or location of atherosclerotic lesions, such as lipid-rich lesions, such as vulnerable plaques, that includes the step of: inserting a Raman spectroscopy probe or catheter probe according to the invention into the lumen of a blood vessel; and optically interrogating the blood vessel wall using Raman spectroscopy at one or more positions in the blood vessel. In one variation, the wavenumber region used for evaluating the blood vessel wall is in the range of approximately 2,600 to 3,200 cm⁻¹ (the “high wavenumber region”) to, for example, evaluate the lipid content of the target. In another variation, the wavenumber region used for evaluating the blood vessel wall is in the range of approximately 200 to 2,000 cm⁻¹ (the “fingerprint region”), such as in the range of approximately 800 to 1,200 cm⁻¹, to, for example, evaluate the calcification of the target. In still another variation, wavenumber regions in each of the high wavenumber region and the fingerprint region (such as in the range of approximately 800 to 1,200 cm⁻¹ for the fingerprint region) are used for analysis in order to evaluate both the lipid content and calcification of the examined target.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A shows a Raman spectrum of PTFE in the Raman fingerprint region (˜200-2,000 cm⁻¹).

FIG. 1B shows a Raman spectrum of PTFE in the “highwave Raman” region (˜2,600-3,200 cm⁻¹).

FIG. 2 shows a windowless side-firing Raman spectroscopy probe embodiment of the invention.

FIG. 3 shows a Raman spectrum of a cholesterol sample collected using a side-firing probe embodiment of the invention as described in FIG. 2.

FIG. 4 shows Raman spectra of cholesterol and cholesterol esters collected using free space optics.

FIG. 5 shows a basket-style intravascular catheter embodiment of the invention.

### DETAILED DESCRIPTION

The present invention provides windowless fiber optic Raman spectroscopy probes such as windowless intravascular catheters for evaluating blood vessel walls. The fiber optic probes of the invention include one or more optical fibers housed in material(s) having a very low Raman scattering cross-section in the wavenumber region used for analysis of a target and being adequately transparent to excitation light delivered via the fiber optics to the target and to Raman-scattered light (inelastically scattered light) collected from the irradiated target in the desired wavenumber range. By very low Raman scattering cross-section, it is meant that the Raman signal of the enclosure material in the wavenumber region used for analysis of the target (sample) is at least substantially non-discemable in the time frame and with the illumination power used to interrogate the sample, for example, within 5 seconds, within 1 second or less. In one embodiment, the Raman signal of the enclosure material in the wavenumber range used for analysis of the target (sample) is at least ten times lower than the target's Raman signal in the wavenumber range used for analysis. In a related embodiment, the Raman signal of the enclosure material in the wavenumber range used for analysis of the target (sample) is at least one-hundred times lower than the target's Raman signal in the wavenumber range used for analysis

In cardiovascular applications of Raman spectroscopy, it is desirable to enclose the fiber optic assembly, for example, catheter or viewing arm thereof, in polymer tubing to protect the probe optics and to provide a smooth catheter surface to navigate delicate arteries. The present invention utilizes materials that are essentially “Raman inactive” in the spectral regions of interest and allow one to launch excitation light through the material and to collect the resulting scattered light through the material. In previous studies where investigators were compelled to have intervening materials between their collection optics and the sample under investigation, attempts were made to subtract the spectrum of the intervening material from that of the investigated material, which can be challenging to accurately perform. Thus, many prior systems relied on optical windows of silica, glass, quartz or other materials that interrupted the general enclosure of the catheter to provide an optical path in and out of the probe.

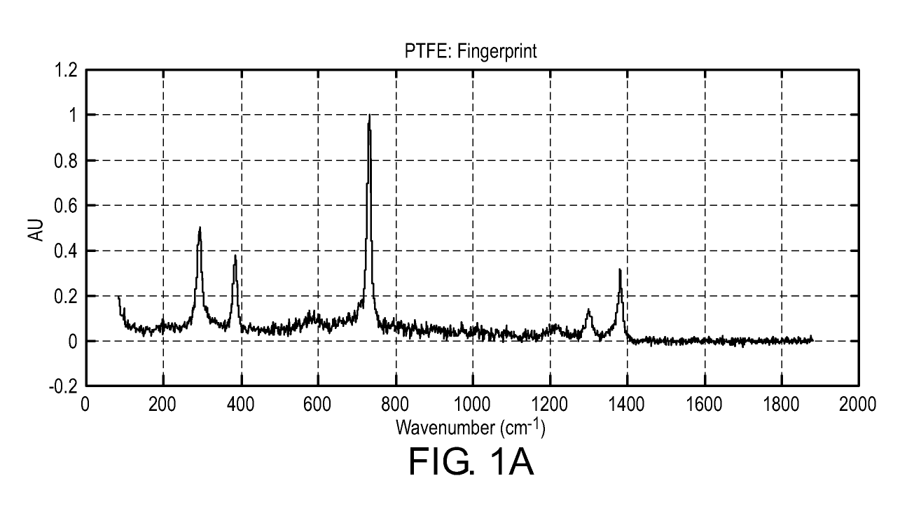

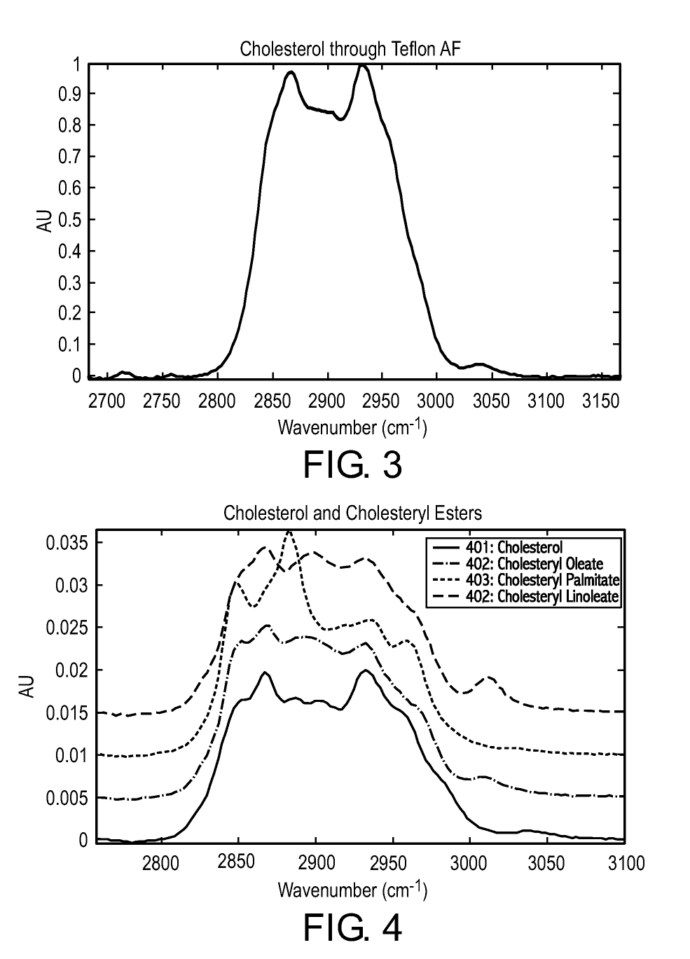

FIG. 1A shows the Raman spectrum of PTFE in the Raman fingerprint region (i.e. 200 to 2,000 cm⁻¹) and FIG. 1B shows the Raman spectrum of PTFE in the “high wavenumber” region (approximately 2,600 to 3200 cm⁻¹) and also above and below the high wavenumber region. The excitation wavelength used to obtain the high wavenumber spectra was 740 nm and that used to obtain the fingerprint region spectra was 830 nm. No discernable Raman spectral features are present in the high wavenumber shifted region (the features in the highwave spectrum lower than 2000 cm⁻¹ are measurement artifacts.) Thus, according to one aspect of the invention, a Raman optical catheter may be constructed by enclosing one or more optical fibers in a fluoropolymer, such as PTFE, and investigating the Raman shifted light of desired samples in the high wavenumber shifted spectral region without contaminating the sample spectra with catheter material artifacts. As shown in FIG. 4, lipid components of lipid-rich atherosclerotic lesions, such as vulnerable plaques, have characteristic Raman spectra within the high wavenumber region. Similarly, in the fingerprint region, there is no substantial Raman signal from 800 to 1,200 cm⁻¹. Since atherosclerotic calcifications show a strong Raman signal at around 960 cm⁻¹, fluoropolymer-enclosed optical probes are also well suited to detecting calcifying or calcified plaques. Hence, the probes and methods of the invention are useful in locating and characterizing lipid-rich lesions, such as vulnerable plaques, in a blood vessel as well as locating and characterizing calcifying or calcified atherosclerotic lesions in a blood vessel and for distinguishing between lipid-rich and calcifying/calcified lesions in a blood vessel.

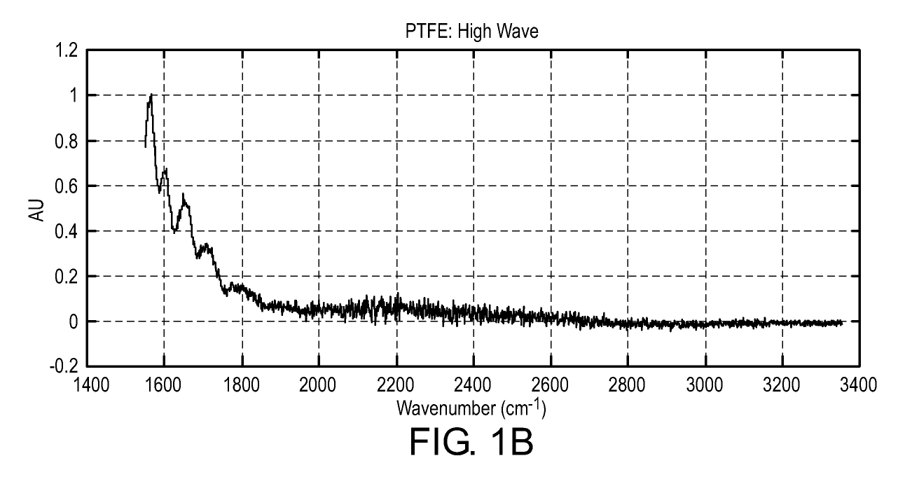

FIG. 2 shows a windowless side-firing Raman spectroscopy probe embodiment of the invention that includes an optical fiber 201 having an angled end face for lateral viewing, which is enclosed in a fluoropolymer tubing 202, for optically interrogating a sample 203. The optical fiber may be enclosed in the fluoropolymer using any method, for example, using heat-shrink PTFE tubing. It should be understood that the enclosures referred to herein may be the outer-most enclosures of at least the viewing portions of optical fibers or optical fiber assemblies of the probes of the invention.

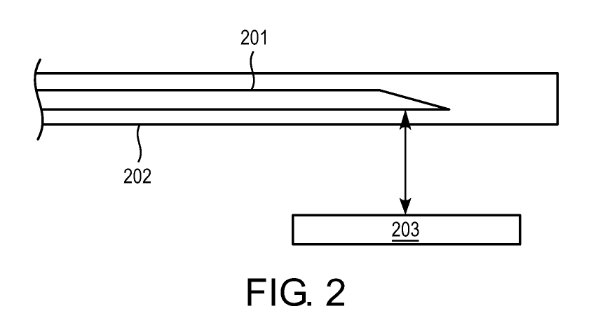

FIG. 3 shows a cholesterol spectrum that was collected using a Teflon AF-enclosed, side-firing probe as shown in FIG. 2. This spectrum is essentially the same as the cholesterol spectrum that was collected with free space optics, which is shown as curve 401 in FIG. 4. Significantly, spectra may be collected through the catheter housing material itself, without the need to construct special viewing windows. Although the results shown here are demonstrated with Teflon AF, other polymer materials exists with very low Raman scattering cross section in a spectral region of interest and may be used to construct catheters. The enclosure material may, for example, include or consist of other polymer materials that at least substantially do not include carbon-hydrogen bonds and which, therefore, can be expected to have an at least substantially non-discernable cross-section in the high wavenumber Raman region. In one embodiment, the polymers used include carbon-carbon bonds, but at least substantially lack carbon-hydrogen bonds. Thus, the polymers may have a carbon backbone. Examples of suitable polymers are polytetrafluoroethylene (PTFE), fluorinated ethylene-propylene (FEP) and perfluoroalkoxy polymer resin (PFA) and amorphous fluoropolymers such as Teflon AF. Amorphous fluoropolymers are provided, for example, by U.S. Pat. Nos. 4,530,569 and 5,076,659, each of which is incorporated by reference herein in its entirety. Advantageously, PTFE and the other fluoropolymers also have very low coefficients of friction which should facilitate non-traumatic interrogation of lumen walls and passage of the catheter/probe though tortuous lumens.

FIG. 4 shows Raman spectra of cholesterol and cholesterol esters collected using free space optics. Specifically, curve 401 is a Raman spectrum for cholesterol, curve 402 is a Raman spectrum for cholesteryl oleate, curve 403 is a Raman spectrum for cholesteryl palmitate and curve 404 is a Raman spectrum for cholesteryl linolenate.

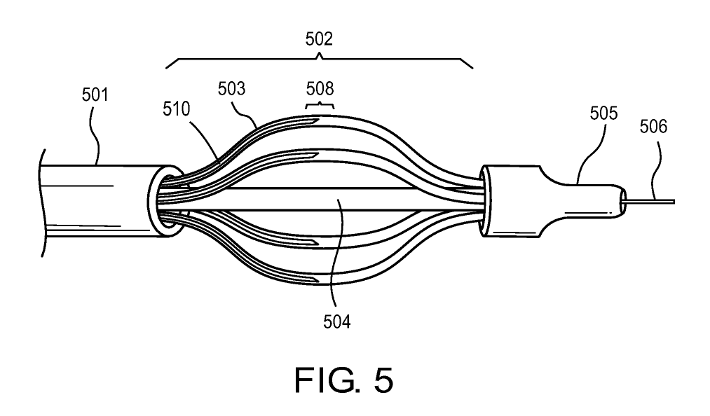

The present invention also provides side/lateral-viewing catheters in which optical components for interrogating the walls of blood vessel lumens are disposed on rods that can be flexed outward toward a blood vessel wall. FIG. 5 shows a basket-style side-viewing optical catheter embodiment of the invention that has a proximal outer shaft 501, a basket section 502 including four probe arms 503 each including one or more side viewing optical fiber assemblies 510, such as an end-angled optical fiber that terminates in or around the apex of the radially extended probe arm (side-viewing portion 508 of the basket section) in order to contact or near a vessel wall so that a Raman spectroscopic evaluation of the vessel wall can be performed. The catheter also includes a distal tip 505 that is connected to a guidewire tube 504, so that the catheter may travel over a guidewire 506, and to the distal end of each probe arm. As shown, the entire probe arm including the side-viewing portion of the optical fiber assembly is enclosed in a fluoropolymer, such as an amorphous fluoropolymer, such as Teflon AF 2400. Any suitable enclosure material as described herein may be used. It has been found that amorphous fluoropolymers have a sufficient rigidity to be used as the main structural element of the rods (arms) of the basket catheter. However, if necessary, each probe arm may also include a structural support element such as spring wire or durable polymer rod (not shown) to support the enclosed fiber optic probe. Radial expansion and contraction of the probe arms of the basket section may be accomplished by contracting and extending the opposite ends of the probe arms, respectively. The guidewire tube, which is attached to the distal tip of the catheter, may be slideable within the catheter thereby permitting said contracting and extending of the opposite ends of the probe with respect to each other, while the proximal ends of the probe arms remain fixed with respect to the proximal outer shaft. Alternatively, a slideable sheath may be provided to control the radial extension of the basket section. Optional radiopaque marker bands may also be provided to aid in visualizing the catheter within a blood vessel.

One related embodiment of the invention provides catheter apparatus for optically interrogating a blood vessel wall, that includes: multiple optical probe rod elements (e.g., 2, 3, 4, 6, or 8) along a central shaft of the catheter and extendable radially outward toward a blood vessel wall from an unextended configuration closer to the longitudinal axis of the catheter and an expandable balloon collectively enclosing the multiple rod elements. The rod elements each include an optical assembly for transmitting and receiving light from the vessel wall lateral to the axis of the catheter while the rod-elements contact or are near the wall. Each of the optical assemblies is in optical communication with at least one optical fiber that is in communication with a light source for illuminating the vessel wall and/or a detector for detecting light received from the vessel wall. The optical assemblies of each rod element may be disposed at or around the middle of a rod element or at or around whatever part of a rod element tends to extend most radially outward. Relative motion of the distal ends and proximal ends of the rods may be used to radially flex the rods outward toward a lumen wall and to radially retract the rods toward the catheter axis.

Another embodiment of the invention provides a catheter apparatus for optically interrogating a blood vessel wall that includes: (1) a rod element portion near the distal end of the catheter comprising multiple rod elements along a central shaft of the catheter and extendable radially outward toward a blood vessel wall from an unextended configuration closer to the longitudinal axis of the catheter, wherein the rod elements each include an optical assembly for transmitting and receiving light from the vessel wall lateral to the axis of the catheter while the rod-elements contact or are near the wall and wherein each of the optical assemblies is in optical communication with at least one optical fiber that is in communication with a light source for illuminating the vessel wall and/or a detector for detecting light received from the vessel wall; and (2) a tip portion of the catheter that extends from the distal end of the rod element portion to the distal end of the catheter, wherein a guidewire conduit or channel extends from within the central shaft of the rod element portion of the catheter distally through the tip portion of the catheter. The guidewire channel or conduit may, for example, open within the rod element portion of the catheter and at or near the distal end of the tip portion of the catheter.

Any suitable sort of side/lateral-viewing optical assembly(ies) may be used and numerous sorts of side viewing optics are known in the art. For example, a 45-deg (or other angle) mirror face or a prism can be used to laterally direct/redirect light from an optical fiber. Similarly, an optical fiber can be provided with an angularly faceted tip to direct and receive light that is off-axis with respect to the fiber, for example, as shown in FIGS. 2 and 5.

In addition to side/laterally-viewing windowless fiber optic Raman probes, the present invention also provides end/front-viewing windowless Raman spectroscopic probe embodiments. For example, at least the end-face of a front-viewing optical fiber may be enclosed by a fluoropolymer material.

The windowless Raman spectroscopy probes of the invention may be used for any purpose or in any application.

One embodiment of the invention provides a method for evaluating the health of blood vessels, for example, by diagnosing, characterizing and/or locating one or more atherosclerotic lesions in a blood vessel, such as an artery, by using a windowless Raman probe as described herein to optically evaluate the properties of a vessel wall at one more locations along the vessel.

A related embodiment of the invention provides a method for diagnosing and/or locating one or more lipid-rich atherosclerotic lesions in a blood vessel, such as a coronary artery of a subject, using a windowless Raman probe as described herein to optically evaluate the properties of a vessel wall at one more locations along the vessel.

Still another related embodiment of the invention provides a method for diagnosing and/or locating one or more vulnerable plaque lesions in a blood vessel, such as a coronary artery of a subject, using a windowless Raman probe as described herein to optically evaluate the properties of a vessel wall at one more locations along the vessel.

In one variation of the method embodiments of the invention, the wavenumber range used to evaluate the blood vessels by Raman spectroscopy is within the range of approximately 2,600 to 3,200 cm⁻¹, i.e., in the high wavenumber region. As shown in FIGS. 3 and 4, various lipid components typical of blood vessel walls and atherosclerotic lesions thereof may be detected and characterized by examining within this wavenumber range.

The invention also provides an integrated system for evaluating the status of a blood vessel wall, for example, for diagnosing and/or locating vulnerable plaque lesions, that includes an windowless Raman probe, such as a catheter, according to the invention, in communication with a light source such as a laser for illuminating a target region of a blood vessel via the catheter and a light analyzer, such as a spectroscope, for analyzing the properties of light received from the target region via the catheter. One or more computers, or computer processors generally working in conjunction with computer accessible memory, may be part of the system for controlling the system and/or for analyzing information obtained by the system.

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A fiber optic Raman spectroscopy probe, comprising:
an optical fiber or optical fiber assembly, said fiber or assembly having a viewing portion for transmitting and receiving light, wherein at least the viewing portion of the fiber or assembly is enclosed in a material having an at least substantially non-discernable Raman scattering signal in one or more preselected wavenumber regions used for analysis of a target and being adequately transparent to excitation light delivered via the optical fiber assembly to the target and to Raman-scattered light collected from the irradiated target in the preselected wavenumber range by the optical fiber assembly.

2. The probe of claim 1, wherein the probe comprises a single optical fiber.

3. The probe of claim 1, wherein the probe comprises at least two optical fibers.

4. The probe of claim 1, wherein the material comprises a polymer.

5. The probe of claim 4, wherein the polymer at least substantially lacks carbon-hydrogen bonds.

6. The probe of claim 4, wherein the polymer comprises at least one fluoropolymer.

7. The probe of claim 6, wherein the fluoropolymer is an amorphous fluoropolymer.

8. The probe of claim 6, wherein the at least one fluoropolymer comprises at least one fluoropolymer selected from the group consisting of polytetrafluoroethylene (PTFE), fluorinated ethylene-propylene (FEP) and perfluoroalkoxy polymer resin (PFA).

9. The probe of claim 1, wherein a preselected wavenumber region is in the range of approximately 2,600 to 3,200 cm⁻¹.

10. The probe of claim 9, wherein the material comprises a polymer at least substantially lacking carbon-hydrogen bonds.

11. The probe of claim 10, wherein the material comprises at least one fluoropolymer.

12. The probe of claim 11, wherein the at least one fluoropolymer comprises at least one fluoropolymer selected from the group consisting of polytetrafluoroethylene (PTFE), fluorinated ethylene-propylene (FEP) and perfluoroalkoxy polymer resin (PFA).

13. The probe of claim 11, wherein the fluoropolymer is an amorphous fluoropolymer.

14. The probe of claim 1, wherein a preselected wavenumber region is in the range of approximately 200 to 2,000 cm⁻¹.

15. An intravascular diagnostic catheter comprising a probe according to claim 1.

16. A catheter apparatus for optically interrogating a blood vessel wall by Raman Spectroscopy, comprising:
multiple rod elements along a central shaft of the catheter and extendable radially outward toward a blood vessel wall from an unextended configuration closer to the longitudinal axis of the catheter, wherein the rod elements each comprise an optical fiber or optical fiber assembly, said fiber or assembly having a viewing portion for transmitting and receiving light from the vessel wall lateral to the axis of the catheter while the rod-elements contact or are near the wall, wherein each of the optical fibers or optical assemblies is in optical communication with one or more of a light source for illuminating the vessel wall and a detector for detecting light received from the vessel wall,
wherein at least the viewing portion of the fiber or assembly is enclosed in a material having an at least substantially non-discemable Raman scattering signal in one or more preselected wavenumber regions used for analysis of a target and being adequately transparent to excitation light delivered via the optical fiber assembly to the target and to Raman-scattered light collected from the irradiated target in the preselected wavenumber range by the optical fiber assembly.

17. The probe of claim 16, wherein the material comprises a polymer.

18. The probe of claim 17, wherein the polymer at least substantially lacks carbon-hydrogen bonds.

20. The probe of claim 19, wherein the material comprises at least one fluoropolymer.

21. The probe of claim 20, wherein the at least one fluoropolymer comprises at least one fluoropolymer selected from the group consisting of polytetrafluoroethylene (PTFE), fluorinated ethylene-propylene (FEP) and perfluoroalkoxy polymer resin (PFA).

22. The probe of claim 20, wherein the fluoropolymer is an amorphous fluoropolymer.

23. The probe of claim 16, wherein a preselected wavenumber region is in the range of approximately 2,600 to 3,200 cm⁻¹.

24. The probe of claim 23, wherein the material comprises at least one fluoropolymer.

25. The probe of claim 24, wherein the fluoropolymer is an amorphous fluoropolymer.
